(19) United States
(12) Patent Application Publication
(10) Pub. No.: US 2026/0325077 A1
(43) Pub. Date: Oct. 22, 2026

(54) SENSOR-BASED ENTITY MONITORING

(71) Applicant: RyuKai Elvin, Vernal, UT (US)

(72) Inventor: RyuKai Elvin, Vernal, UT (US)

(21) Appl. No.: 19/468,631

(22) Filed: Feb. 3, 2026

Related U.S. Application Data
(60) Provisional application No. 63/753,068, filed on Feb. 3, 2025.

(51) Int. Cl. A61B 5/00; B60H 1/00; B60W 50/14; H04M 1/72409; H04M 1/72424; H04M 1/72454

(52) U.S. Cl. CPC A61B 5/6893; A61B 5/4842; B60H 1/0073; B60H 1/00742; B60W 50/14; H04M 1/724098; H04M 1/72454; A61B 2560/0443; B60W 2540/221; B60W 2756/10; H04M 1/72424

(57) ABSTRACT

Systems and methods for remote monitoring of users at a vehicle are disclosed herein. A system may initiate a monitoring session by generating one or more commands configured to activate a sensor module at a detachable monitoring system. The system may obtain sensor data comprising one or more health indicative metrics measured by the detachable monitoring system using the sensor module. Based on the sensor data, the system may generate a set of commands comprising a command configured to actuate one or more components of the vehicle to modify an operational state of the vehicle and/or a command configured to generate an alert. The system may transmit the command(s).

### CROSS-REFERENCE TO RELATED APPLICATIONS

This application claims the priority benefit of U.S. Provisional Application No. 63/753,068, filed Feb. 3, 2025, entitled “BABYSAFE,” which is incorporated herein by reference in its entirety.

### BACKGROUND

In modern systems, sensors are integral in capturing, monitoring, and transmitting data from the physical environment to digital systems. Sensor devices, which can detect a wide range of physical phenomena such as temperature, pressure, motion, or chemical composition, are foundational to applications spanning industrial automation, healthcare, transportation, and consumer electronics.

### SUMMARY

For purposes of this summary, certain aspects, advantages, and novel features are described herein. It is to be understood that not all such advantages necessarily may be achieved in accordance with any particular embodiment. Thus, for example, those skilled in the art will recognize that the present technology may be embodied or carried out in a manner that achieves one advantage or group of advantages as taught herein without necessarily achieving other advantages as may be taught or suggested herein.

In some implementations, the techniques described herein relate to a system for remote monitoring of users at a vehicle, the system comprising: one or more processors; and one or more memories configured to store instructions that, when executed by the one or more processors, perform operations comprising: obtaining a location of a detachable monitoring system in relation with the vehicle, wherein the detachable monitoring system is configured to measure, via a sensor module of the detachable monitoring system, one or more health indicative metrics of a secondary user while a secondary user is engaged with the detachable monitoring system; responsive to determining that (i) the detachable monitoring system is located within the vehicle and (ii) that the secondary user is not actively monitored by a primary user, initiating a monitoring session by generating one or more commands configured to activate the sensor module at the detachable monitoring system; obtaining sensor data comprising one or more health indicative metrics measured by the detachable monitoring system using the sensor module; based on the sensor data, generating a set of commands comprising (a) a first command configured to actuate one or more components of the vehicle to modify an operational state of the vehicle, and (b) a second command configured to generate an alert at a paired user device of the primary user or to one or more additional entities within a threshold distance of the vehicle; and transmitting the first command to the one or more components of the vehicle and the second command to the paired user device of the primary user or to one or more additional entities.

In some implementations, the techniques described herein relate to a system, wherein determining that the secondary user is not actively monitored by the primary user comprises (a) detecting that a paired user device of a primary user is outside of a predetermined distance from the vehicle or (b) that the vehicle is in operation.

In some implementations, the techniques described herein relate to a system, wherein the instructions further cause the one or more processors to perform operations comprising: detecting that the paired user device of a primary user is outside of a predetermined distance from the vehicle; and responsive to detecting that the paired user device is outside of the predetermined distance, modifying the operational state of the vehicle to maintain temperature of the vehicle within an upper threshold temperature and a lower threshold temperature.

In some implementations, the techniques described herein relate to a system, wherein generating the set of commands comprises: generating, using the sensor data, a vector for input to a machine learning model trained to generate the set of commands; and inputting the vector into the machine learning model to obtain the set of commands.

In some implementations, the techniques described herein relate to a system, wherein generating the set of commands based on the sensor data comprises: determining that a subset of health indicative metrics matches a pattern of declining health from a plurality of patterns; identifying the first command configured to actuate one or more components of the vehicle to modify the operational state of the vehicle from a plurality of commands; and identifying the second command configured to generate the alert at the paired user device of the primary user or to one or more additional entities within the threshold distance of the vehicle from the plurality of commands.

In some implementations, the techniques described herein relate to a system, wherein the instructions further cause the one or more processors to perform operations comprising: determining that the primary user is attempting to operate the vehicle; determining (i) whether the detachable monitoring system is within a vehicle and (ii) whether the secondary user is engaged with the detachable monitoring system; and transmitting a command configured to generate an alert at the paired user device responsive to determining that the detachable monitoring system is not within a vehicle or that the secondary user is not engaged with the detachable monitoring system.

In some implementations, the techniques described herein relate to a method for remote monitoring of users at a vehicle, the method comprising: responsive to determining that a secondary user is not actively monitored by a primary user, initiating a monitoring session by generating one or more commands configured to activate a sensor module at a detachable monitoring system; obtaining sensor data comprising one or more health indicative metrics measured by the detachable monitoring system using the sensor module; based on the sensor data, generating a set of commands comprising (a) a first command configured to actuate one or more components of the vehicle to modify an operational state of the vehicle, and (b) a second command configured to generate an alert at a paired user device of the primary user or to one or more additional entities within a threshold distance of the vehicle; and transmitting the first command to the one or more components of the vehicle and the second command to the paired user device of the primary user or to one or more additional entities within a threshold distance of the vehicle.

In some implementations, the techniques described herein relate to a method, further comprising: obtaining a location of a detachable monitoring system in relation with the vehicle.

In some implementations, the techniques described herein relate to a method, wherein determining that the secondary user is not actively monitored by the primary user comprises (a) detecting that a paired user device of a primary user is outside of a predetermined distance from the vehicle or (b) that the vehicle is in operation.

In some implementations, the techniques described herein relate to a method, further comprising: detecting that the paired user device of a primary user is outside of a predetermined distance from the vehicle; and responsive to detecting that the paired user device is outside of the predetermined distance, modifying the operational state of the vehicle to maintain temperature of the vehicle within an upper threshold temperature and a lower threshold temperature.

In some implementations, the techniques described herein relate to a method, wherein generating the set of commands comprises: generating, using the sensor data, a vector for input to a machine learning model trained to generate the set of commands; and inputting the vector into the machine learning model to obtain the set of commands.

In some implementations, the techniques described herein relate to a method, wherein generating the set of commands based on the sensor data comprises: determining that a subset of health indicative metrics matches a pattern of declining health from a plurality of patterns; identifying the first command configured to actuate one or more components of the vehicle to modify the operational state of the vehicle from a plurality of commands; and identifying the second command configured to generate the alert at the paired user device of the primary user or to one or more additional entities within the threshold distance of the vehicle from the plurality of commands.

In some implementations, the techniques described herein relate to a method, further comprising: determining that the primary user is attempting to operate the vehicle; determining (i) whether the detachable monitoring system is within a vehicle and (ii) whether the secondary user is engaged with the detachable monitoring system; and transmitting a command configured to generate an alert at the paired user device responsive to determining that the detachable monitoring system is not within a vehicle or that the secondary user is not engaged with the detachable monitoring system.

In some implementations, the techniques described herein relate to a method, wherein the one or more additional entities comprises an emergency services entity.

In some implementations, the techniques described herein relate to one or more non-transitory, computer-readable storage media storing instructions that when executed by one or more processors cause the one or more processors to perform operations comprising: responsive to determining that a detachable monitoring system is located within a vehicle, initiating a monitoring session by generating one or more commands configured to activate a sensor module at the detachable monitoring system; obtaining sensor data comprising one or more health indicative metrics of a secondary user measured by the detachable monitoring system using the sensor module; based on the sensor data, generating a set of commands comprising (a) a first command configured to actuate one or more components of the vehicle to modify an operational state of the vehicle, and (b) a second command configured to generate an alert at a paired user device of a primary user or to one or more additional entities within a threshold distance of the vehicle; and transmitting the first command to the one or more components of the vehicle and the second command to the paired user device of the primary user or to one or more additional entities.

In some implementations, the techniques described herein relate to one or more non-transitory, computer-readable storage media, wherein the instructions further cause the one or more processors to perform operations comprising: obtaining a location of a detachable monitoring system in relation with the vehicle, wherein the detachable monitoring system is configured to measure, via a sensor module of the detachable monitoring system, one or more health indicative metrics of a secondary user while a secondary user is engaged with the detachable monitoring system.

In some implementations, the techniques described herein relate to one or more non-transitory, computer-readable storage media, wherein the instructions further cause the one or more processors to perform operations comprising: determining that the secondary user is not actively monitored by a primary user based on detecting (a) that a paired user device of a primary user is outside of a predetermined distance from the vehicle or (b) that the vehicle is in operation; and responsive to determining that the secondary user is not actively monitored by a primary user, activating a sensor module at the detachable monitoring system.

In some implementations, the techniques described herein relate to one or more non-transitory, computer-readable storage media, wherein the instructions further cause the one or more processors to perform operations comprising: detecting that the paired user device of a primary user is outside of a predetermined distance from the vehicle; and responsive to detecting that the paired user device is outside of the predetermined distance, modifying the operational state of the vehicle to maintain temperature of the vehicle within an upper threshold temperature and a lower threshold temperature.

In some implementations, the techniques described herein relate to one or more non-transitory, computer-readable storage media, wherein generating the set of commands based on the sensor data comprises: determining that a subset of health indicative metrics matches a pattern of declining health from a plurality of patterns; identifying the first command configured to actuate one or more components of the vehicle to modify the operational state of the vehicle from a plurality of commands; and identifying the second command configured to generate the alert at the paired user device of the primary user or to one or more additional entities within the threshold distance of the vehicle from the plurality of commands.

In some implementations, the techniques described herein relate to one or more non-transitory, computer-readable storage media, wherein the instructions further cause the one or more processors to perform operations comprising: determining that the primary user is attempting to operate the vehicle; determining (i) whether the detachable monitoring system is within a vehicle and (ii) whether the secondary user is engaged with the detachable monitoring system; and transmitting a command configured to generate an alert at the paired user device responsive to determining that the detachable monitoring system is not within a vehicle or that the secondary user is not engaged with the detachable monitoring system.

Various other aspects, features, and advantages of the system will be apparent through the detailed description and the drawings attached hereto. It is also to be understood that both the foregoing general description and the following detailed description are examples, and not restrictive of the scope of the disclosure. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification, “a portion” refers to a part of, or the entirety of (i.e., the entire portion), a given item (e.g., data), unless the context clearly dictates otherwise.

### BRIEF DESCRIPTION OF THE DRAWINGS

FIG. 1 shows an illustrative system for remote monitoring of users at a vehicle, in accordance with one or more embodiments of this disclosure.

FIG. 2 illustrates an exemplary data structure comprising sensor data, in accordance with one or more embodiments of this disclosure.

FIG. 3A illustrates an exemplary detachable monitoring system, in accordance with one or more embodiments of the disclosure.

FIG. 3B illustrates an exemplary paired user device, in accordance with one or more embodiments of the disclosure.

FIG. 4 illustrates an exemplary data structure comprising commands, in accordance with one or more embodiments of the disclosure.

FIG. 5 illustrates a computing system that can be used for remote monitoring of users at a vehicle, in accordance with one or more embodiments of this disclosure.

FIG. 6 is a flowchart of operations for remote monitoring of users at a vehicle, in accordance with one or more embodiments of this disclosure.

### DETAILED DESCRIPTION

In the following description, for the purposes of explanation, numerous specific details are set forth in order to provide a thorough understanding of the disclosed embodiments. It will be appreciated, however, by those having skill in the art, that the embodiments may be practiced without these specific details or with an equivalent arrangement. In other cases, well-known models and devices are shown in block diagram form in order to avoid unnecessarily obscuring the disclosed embodiments. It should also be noted that the methods and systems disclosed herein are also suitable for applications unrelated to source code programming.

### Introduction

The field of health monitoring through electronic sensors has advanced significantly in recent years, enabling continuous, non-invasive tracking of vital signs and physiological parameters. Modern sensor technologies, including pulse oximeters, temperature sensors, accelerometers, and respiratory monitors, can detect a wide range of health indicators in real-time. These sensors, when integrated with microprocessors and wireless communication systems, enable automated monitoring systems capable of detecting anomalies and triggering appropriate responses without requiring constant human supervision.

However, implementing comprehensive health and safety monitoring within the context of child car seats presents unique technical challenges. The automotive environment introduces variables such as temperature fluctuations, vehicle motion, varying ambient conditions, and the need for integration with vehicle systems. Furthermore, the vulnerable population being monitored (i.e., infants and young children) cannot communicate distress effectively, making reliable automated detection critical. A significant technical difficulty lies in the diversity of problem types that must be simultaneously addressed such as physiological emergencies such as positional asphyxiation and sleep apnea, environmental hazards including dangerous temperature extremes, and situational risks such as a child being inadvertently left unattended in a vehicle. Each problem type requires different sensing modalities and detection algorithms.

Equally challenging is the variety of mitigation actions that must be coordinated in response to detected problems. These actions may include alerting caregivers through mobile notifications, activating vehicle climate control systems, triggering audible alarms, contacting emergency services, or providing vehicle location information. Ensuring that detection systems and response mechanisms mesh appropriately such that the correct mitigation action is triggered for each specific problem type with appropriate urgency and without false positives that could desensitize caregivers requires sophisticated system integration.

Common issues that such a system should address include: positional asphyxiation and sleep apnea which can cause serious injury or death and often go undiagnosed in children; dangerous vehicle temperature conditions when children are left unattended in hot or cold environments; caregiver incapacitation scenarios where a parent or guardian experiences a medical emergency and cannot assist their child; and the practical challenge of locating a vehicle in parking areas without triggering loud alarms that might disturb a sleeping infant.

What is needed is a detachable monitoring system (e.g., integrated child car seat) that combines multiple sensor types for comprehensive health and environmental monitoring and a system that incorporates intelligent processing to distinguish between different problem types and severity levels and coordinates appropriate mitigation responses ranging from caregiver notifications to emergency service contact and vehicle component actuation, such as climate control activation. Systems are disclosed herein for remote monitoring of users at a vehicle.

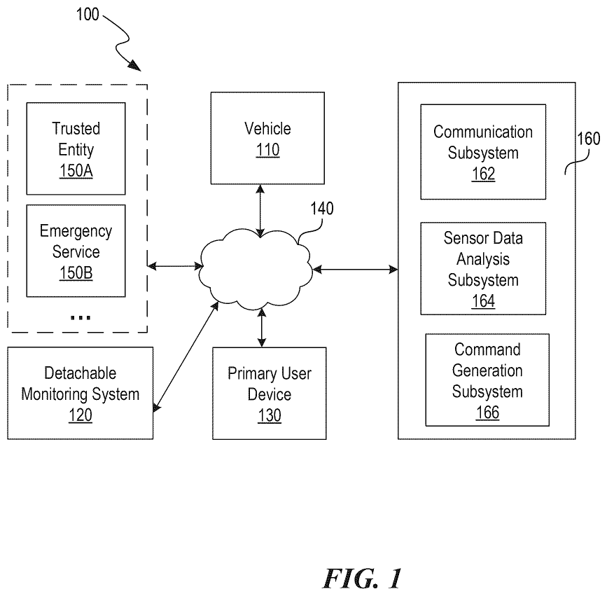

Environment 100 of FIG. 1 is an illustrative system that can be used for remote monitoring of entities (e.g., children, infants), including when the entity is within or proximate to a vehicle. As an example, environment 100 may be used to monitor an entity such as a child engaged in a detachable monitoring system 120, such as a car seat equipped with sensors. As referred to herein, an entity, as described herein, may include a child, infant, pet, or other human, whose physiological or behavioral state can be monitored when engaged with a detachable monitoring system. The detachable monitoring system 120 may be implemented in various forms, such as a car seat equipped with integrated sensors capable of measuring health indicative metrics including, but not limited to, temperature, heart rate, and movement.

As referred to herein, engagement of the entity at the detachable monitoring system 120 may be defined as the entity being physically positioned within or upon the monitoring system, thereby enabling continuous data acquisition. In one embodiment, the detachable monitoring system may comprise a car seat equipped with integrated sensors, wherein engagement is established when, for example, a child is seated in the car seat and the safety belt or harness is secured. The closure of the safety restraint may be detected by a sensor, such as a buckle switch or pressure sensor, thereby confirming engagement and activating the monitoring functions. In alternative embodiments, the detachable monitoring system may be implemented as a wearable device, such as a ring, bracelet, or band, which is engaged by the entity wearing the device. Engagement in this context may be detected by capacitive touch sensors, skin contact sensors, or biometric authentication modules embedded within the wearable, ensuring that the device is in direct contact with the entity's skin. According to one implementation, upon engagement, the detachable monitoring system 120 may initiate acquisition of physiological or behavioral metrics, such as heart rate, temperature, movement, or other health indicative parameters, and transmits the data to a paired user device or remote server for further processing and alert generation.

Entity monitoring system 160 of environment 100 may execute instructions for remote monitoring of an entity at the detachable monitoring system 120. Entity monitoring system 160 may include several subsystems, each configured to perform one or more steps of the methods described herein, such as communication subsystem 162, sensor data analysis subsystem 164, and command generation subsystem 166. Entity monitoring system 160 may include software, hardware, or a combination of the two. For example, entity monitoring system 160 may be hosted on a physical server or a virtual server that is running on a physical computer system. In some embodiments, entity monitoring system 160 may be configured on the detachable monitoring system 120 itself, on the vehicle 110, and/or the user device (e.g., a laptop computer, a smartphone, a desktop computer, an electronic tablet, a virtual reality/mixed reality device, or another suitable user device) of a paired user such as a parent, guardian, etc.

The entity monitoring system 160 may receive sensor metrics from the detachable monitoring system 120 and may use the sensor metrics to detect anomalous behavior or patterns of the entity. If the obtained health indicative metrics match or otherwise indicate a pattern of declining health (e.g., high temperature, low temperature, etc.), the entity monitoring system 160 may generate commands to modify the operational state of the vehicle and/or alert a user. In one example, based on the type of pattern, the system may determine which command from a hierarchy of commands or what devices to alert from a plurality of devices. In the example of FIG. 1, the entity monitoring system 160 may alert a paired user, such as via primary user device 130.

A paired user, as referred to herein, may be a primary user such as a parent, legal guardian, or other responsible individual, who is associated with a primary user device 130. The primary user device 130 may comprise a smartphone, tablet, wearable device, or other portable electronic device configured to communicate with the detachable monitoring system via wired or wireless connections. The primary user device is operable to receive sensor data, alerts, and commands from the monitoring system, thereby enabling the paired user to remotely monitor and respond to the state of the entity engaged with the monitoring system.

Alternatively, or additionally, the entity monitoring system 160 may alert a trusted entity 150A. A trusted entity 150A, as referred to herein, may be an individual or party designated by the primary user to receive alerts, notifications, or data from the detachable monitoring system. The trusted entity may include a relative, friend, caregiver, or other person whom the primary user has explicitly authorized through the system's configuration interface. Authorization may be established via secure pairing protocols, user account management, or multi-factor authentication, ensuring that only approved individuals are able to access sensitive information or respond to alerts generated by the monitoring system.

Alternatively, or additionally, the entity monitoring system 160 may alert an emergency service 150B. An emergency service 150B, as referred to herein, may include public safety organizations or personnel such as firefighters, emergency medical technicians (EMTs), law enforcement officers, or other first responders. In some implementations, the entity monitoring system may be configured to transmit alerts, sensor data, or location information directly to emergency service entities in response to detected critical events or health emergencies involving the monitored entity. Communication with emergency services may be established via secure wireless protocols, integration with public safety networks, or automated dispatch systems, thereby enabling rapid intervention and support when urgent assistance is required.

As described herein, communication subsystem 162 of entity monitoring system 160 may be used to obtain data from the detachable monitoring system 120. Communication subsystem 162 may include software components, hardware components, or a combination of both. For example, communication subsystem 162 may include a network card (e.g., a wireless network card and/or a wired network card) that is associated with software to drive the card. Communication subsystem 162 may pass at least a portion of the data, or a pointer to the data in memory, to other subsystems such as sensor data analysis subsystem 164, and/or command generation subsystem 166.

The communication subsystem 162 may obtain a location of a detachable monitoring system in relation with the vehicle. In one embodiment, the detachable monitoring system may be equipped with a global positioning system (GPS) module that transmits its geographic coordinates to the communication subsystem, which then compares these coordinates to the known location of the vehicle. Alternatively, the system may utilize short-range wireless communication protocols, such as Bluetooth Low Energy (BLE) or ultra-wideband (UWB), to determine proximity or relative position by measuring signal strength, time-of-flight, or angle-of-arrival between the monitoring system and the vehicle's onboard receiver. In another embodiment, radio-frequency identification (RFID) tags or near-field communication (NFC) modules may be integrated into the monitoring system and vehicle, enabling the communication subsystem 162 to detect when the monitoring system is within a predefined range or physically docked. Additionally, sensor fusion techniques may be employed, combining data from accelerometers, gyroscopes, and magnetometers to infer the monitoring system's position relative to the vehicle's interior.

The communication subsystem 162 may obtain health metrics from the detachable monitoring system 120. As described herein, in the example where the entity monitoring system is hosted on the detachable monitoring system 120, the entity monitoring system 120 may simply access the health metrics locally. Alternatively, if the entity monitoring system is hosted on the user device or vehicle, the entity monitoring system 160 may obtain sensor data comprising one or more health indicative metrics measured by the detachable monitoring system 120 (e.g., using a sensor module) via network 140. In some implementations, in order to conserve energy, the detachable monitoring system may be in a default deactivated state in which a subset (or all) of the sensors are deactivated. Responsive to determining that the detachable monitoring system is located within the vehicle and/or that the entity (also referred to herein as a secondary user) is not actively monitored by a primary user, the entity monitoring system may initiate a monitoring session by generating one or more commands configured to activate the sensor module at the detachable monitoring system 120.

In one implementation, detecting that a paired user device of a primary user is outside of a predetermined distance from the vehicle may be accomplished using wireless communication protocols such as Bluetooth Low Energy (BLE), Wi-Fi, or cellular connectivity. The entity monitoring system 160 may periodically measure the signal strength or calculates the proximity between the primary user device and the vehicle's onboard receiver. If the measured distance exceeds a predefined threshold, such as 10 meters, the entity monitoring system 160 determines that the primary user device is no longer in close proximity to the vehicle. Alternatively, the system may utilize GPS data from both the vehicle and the primary user device to calculate their relative positions and confirm separation beyond the predetermined distance. Detection that the vehicle is in operation may be achieved by monitoring vehicle status signals, such as ignition state, engine activity, or movement detected by onboard sensors (e.g., accelerometers or speed sensors). In some implementations, when either condition (e.g., primary user device being outside the threshold distance or the vehicle being in operation) is met, the system may initiate a monitoring session, activate safety protocols, or transmit alerts to the primary user and other authorized entities to ensure the safety of the monitored entity.

In one implementation, responsive to determining that the primary user device has exceeded the specified distance threshold, the system may automatically modify the operational state of the vehicle to maintain the vehicle temperature within defined upper and lower threshold values. Specifically, the operational state may include activation or adjustment of the vehicle's climate control subsystem, such as air conditioning, heating, or ventilation, to ensure that the internal temperature remains within a safe and comfortable range for any remaining occupants or sensitive items. This automated climate control response is particularly advantageous in scenarios where children, pets, or temperature-sensitive cargo are present, as it helps prevent exposure to extreme temperatures when the primary user is not in close proximity to the vehicle.

The predetermined proximity threshold for detecting whether a paired user device is sufficiently close to the vehicle may be dynamically set according to the vehicle's type and size, which can be determined during initial system setup. For instance, larger vehicles such as vans, SUVs, or buses may require a greater threshold distance to account for their increased physical dimensions and the extended range over which wireless signals can be reliably detected. Conversely, smaller vehicles such as sedans or compact cars may utilize a shorter proximity threshold, reflecting their reduced footprint and cabin size. The system may automatically identify the vehicle type and size using onboard vehicle identification data, such as VIN, make, model, and year, or through manufacturer databases. This information enables the system to select an appropriate proximity threshold that ensures accurate detection of user presence relative to the vehicle, thereby optimizing safety protocols and minimizing false positives or negatives in user-device association.

In one implementation, responsive to detecting that the detachable monitoring system 120 is outside of the vehicle based on the location data, such as when the monitoring system is positioned above, in front of, or otherwise not within the vehicle's interior, the entity monitoring system 160 may generate one or more commands to prevent vehicle operation. For example, the system may transmit a command to the vehicle's control module to inhibit engine start, disengage the transmission, or activate a parking brake, thereby ensuring that the vehicle cannot be driven until the detachable monitoring system is properly positioned within the vehicle. This functionality may be particularly advantageous in scenarios where the monitoring system is intended to ensure the safety of a child, pet, or other vulnerable entity, as it prevents accidental vehicle movement when the monitoring system is not engaged or present.

In some implementations, the entity monitoring system 160 is configured to determine that the primary user (e.g., parent or guardian controlling the paired device) is attempting to operate the vehicle by monitoring vehicle ignition signals, key fob activity, or user interface interactions. Upon detecting such an attempt, the system further determines whether the detachable monitoring system is within the vehicle and whether the entity is engaged with the detachable monitoring system (e.g., by analyzing location data, seat occupancy sensors, and user engagement metrics). If the system determines that the detachable monitoring system 120 is not within the vehicle or that the secondary user is not engaged with the detachable monitoring system, it will transmit a command configured to generate an alert at the paired user device 130. This alert may be delivered via push notification, audible alarm, or visual indicator on the user's smartphone or other paired device, thereby ensuring that the primary user is immediately informed of any safety-critical conditions before vehicle operation commences.

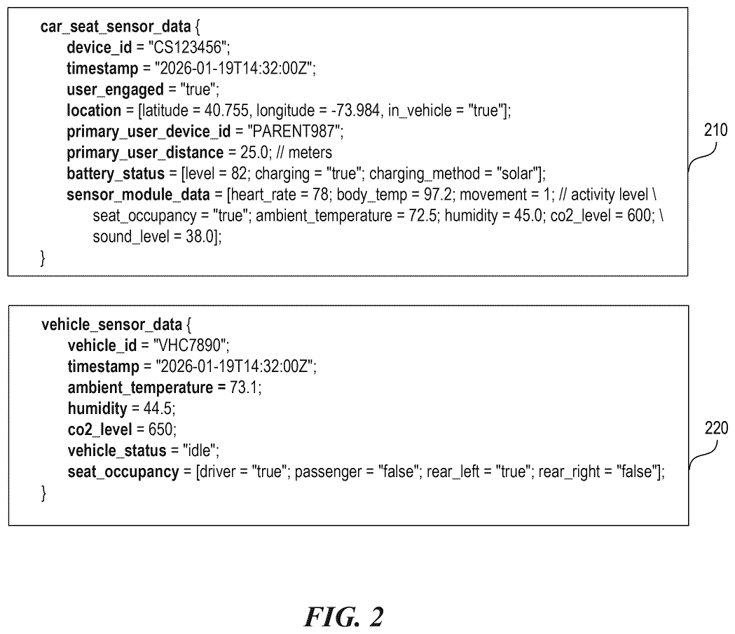

As described herein, the system may initiate a monitoring session at a detachable monitoring system 120. The detachable monitoring system 120 may be configured to measure, via a sensor module of the detachable monitoring system, one or more health indicative metrics of a secondary user while a secondary user is engaged with the detachable monitoring system. For example, FIG. 2 illustrates an exemplary data structure 210 comprising sensor data, in accordance with one or more embodiments of this disclosure.

The exemplary data structure 210 may identify the unique device identifier of the detachable entity monitoring system 120, e.g., “device_id” “CS123456.” The data structure may further include a timestamp (“2026-01-19T14:32:00Z”) and may confirm user engagement (“user_engaged =true”), indicating that the occupant is properly positioned and the seat is actively monitoring. In the example of FIG. 2, the exemplary data structure 210 further includes location data, including latitude (40.755), longitude (−73.984), and an “in_vehicle” status (“true”), verify that the car seat is installed within a vehicle. The primary user device (“PARENT987”) is indicated to be detected at a distance of 25.0 meters from the car seat, which may trigger safety protocols if the primary user is outside a predetermined proximity threshold. The system's battery status may reflect an 82% charge level, with active charging via a solar module, ensuring operational continuity. Sensor module data further include physiological metrics such as heart rate (78 bpm), body temperature (97.2° F.), and activity level (1), as well as environmental parameters: seat occupancy (“true”), ambient temperature (72.5° F.), humidity (45.0%), carbon dioxide concentration (600 ppm), and sound level (38.0 dB).

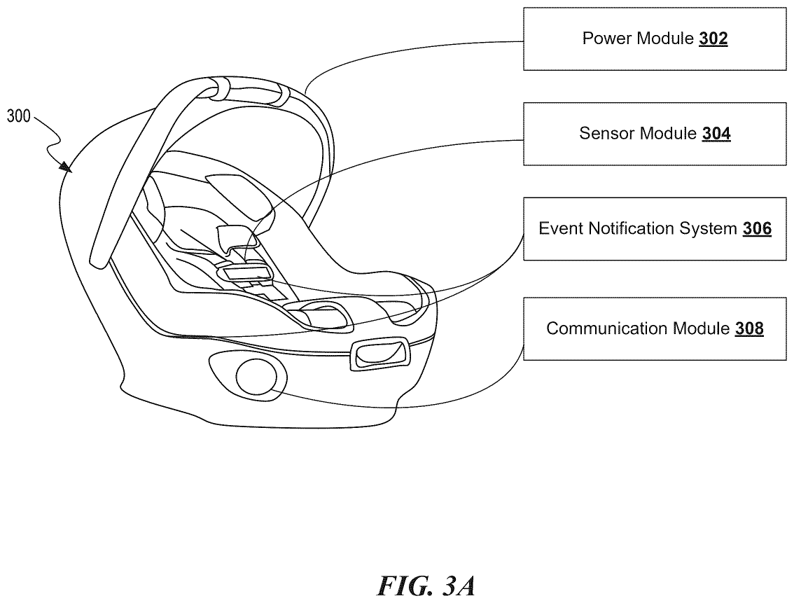

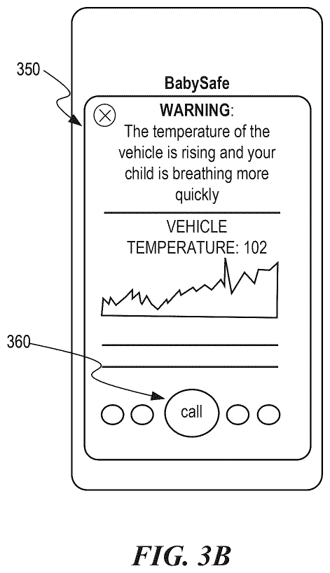

FIG. 3A illustrates an exemplary detachable monitoring system (e.g., detachable monitoring system 120), in accordance with one or more embodiments of the disclosure. The exemplary detachable monitoring system 300 may measure, using sensors disposed thereon, the metrics of the data structure, e.g., data structure 210. It should be noted that the system is not limited to any specific module or subsystem configuration; rather, it may include various subsystems and components as required for particular implementations.

The detachable monitoring system 300 may include a power module 302 configured to supply electrical power to the integrated sensors and electronic components. The power module may be charged via multiple methods, including a top-mounted solar panel for ambient energy harvesting, direct connection to a vehicle power outlet, or a standard electrical outlet. The system may further incorporate a lights power module, which provides illumination for status indication or user interaction. The solar panel is positioned to maximize exposure to sunlight, thereby supporting continuous operation and reducing reliance on external charging sources.

A communication module 308 may be provided within the detachable monitoring system 300 and may enable data transmission between the car seat, the entity monitoring system 160, and/or paired user devices. The communication module may be configured for both wired and wireless connectivity, including but not limited to Bluetooth, Wi-Fi, or direct cable connections to the vehicle's onboard systems or user devices. This module facilitates the secure transfer of sensor data, operational metrics, and event notifications, allowing real-time monitoring and remote control functionalities.

The sensor module 304 may comprise a variety of sensors, such as heart rate monitors, temperature sensors, pressure sensors for seat occupancy detection, accelerometers for movement and activity level assessment, ambient environmental sensors (e.g., humidity, CO₂ concentration, sound level), and biometric sensors for user identification. These sensors collectively enable comprehensive monitoring of the occupant's physiological state and the surrounding environment. Additionally, the detachable monitoring system 300 may include an event notification subsystem 306, which is operable to generate visual or auditory alerts in response to detected events. For example, the subsystem may flash lights or visually emphasize the sides of the car seat, or display notifications on an integrated screen, thereby providing immediate feedback to the user or alerting nearby individuals to critical conditions.

In some implementations, the entity monitoring system 160 may receive, via the communication subsystem 162, sensor data from the vehicle 110 along with the data from the detachable monitoring system 120. Data structure 220 includes a vehicle sensor data record storing a set of environmental and occupancy parameters collected by the vehicle identified by “vehicle_id” “VHC 7890.” The data structure may include a timestamp “2026-01-19T14:32:00Z,” an ambient temperature of 73.1 °F and a relative humidity of 44.5%, e.g., reflecting the internal climate conditions of the vehicle cabin. The data structure 220 may further include a carbon dioxide (CO₂) concentration measured at 650 ppm, providing an indicator of air quality and ventilation status. Data structure 220 may further include a vehicle status identified as “idle,” signifying that the engine is not actively engaged in propulsion. Seat occupancy data may be captured for multiple seating positions, with the driver seat and rear left seat occupied (“true”), while the passenger seat and rear right seat are unoccupied (“false”).

The communication subsystem 162 of the entity monitoring system 160 may clean, parse, and/or filter the data from the vehicle and/or the detachable monitoring system 120. For example, the communication subsystem 162 of the entity monitoring system 160 may be configured to receive raw sensor data streams from both the vehicle 110 and the detachable monitoring system 120. Upon receipt, the subsystem may implement a series of data processing operations to ensure the integrity, relevance, and usability of the information. Data cleaning may involve the removal of corrupted, incomplete, or duplicate records, as well as the correction of outlier values that fall outside expected operational ranges. Parsing operations may include the extraction and structuring of key data fields (e.g., such as timestamps, device identifiers, sensor readings, and status indicators) into standardized formats suitable for downstream analysis. Filtering processes may be applied to isolate data points of interest, such as health indicative metrics, environmental parameters, or occupancy status, while discarding extraneous or non-essential information. The communication subsystem 162 may pass the data, or a pointer in memory to the data, to the sensor data analysis subsystem 164. The sensor data analysis subsystem 164 may be configured to analyze the sensor data to identify anomalous physiological patterns. Based on the sensor data, the sensor data analysis subsystem 164 may generate a set of commands comprising a first command configured to actuate one or more components of the vehicle to modify an operational state of the vehicle, and a second command configured to generate an alert at a paired user device of the primary user or to one or more additional entities within a threshold distance of the vehicle.

For example, the sensor data analysis subsystem 164 may generate, using the sensor data, a vector for input to a machine learning model trained to detect anomalous physiological or behavioral patterns. The sensor data analysis subsystem 164 may then input the vector into the machine learning model to obtain an output indicating whether the entity (e.g., secondary user) such as a child is exhibiting typical health metrics and/or behavior, or if the child is exhibiting health metrics corresponding to an event such as high temperature, e.g., heatstroke, low temperature, e.g., hypothermia, etc. The sensor data may include physiological metrics, environmental conditions, and occupancy status, which are collectively processed to determine the appropriate system response. For example, the machine learning model may output an identifier or code corresponding to a specific pattern of declining health or physiological state.

The identifier or code may be passed to the command generation subsystem 166 where the command generation subsystem 166 may use the identifier or code to identify commands for modifying an operational state of the vehicle and/or generating an alert. As described herein, a first command may be generated by the system and configured to actuate one or more components of the vehicle in order to modify the operational state of the vehicle. The command can be transmitted from the entity monitoring system to the vehicle's control module via a secure communication channel, such as a CAN bus interface, Bluetooth, or Wi-Fi connection. Upon receipt, the vehicle's electronic control unit (ECU) may interpret the command and initiate the corresponding actuation sequence. For example, the first command may instruct the ECU to activate the climate control system, thereby lowering the cabin temperature; to engage or disengage the vehicle's door locks; to inhibit engine start by disabling the ignition circuit; or to activate the parking brake to prevent unintended movement. The command may also be configured to adjust seat positions, enable interior lighting, or trigger warning indicators on the dashboard.

Similarly, a command for generating an alert may be produced by the entity monitoring system 160 in response to sensor data indicating a potentially hazardous or abnormal condition within the vehicle environment. Upon execution, the command may trigger or otherwise initiate a one or multi-step alert protocol designed to notify relevant parties and facilitate rapid intervention. For example, the command may trigger the transmission of a push notification, SMS, or automated voice call to the primary user's mobile device, providing real-time information about the detected risk, such as elevated cabin temperature, abnormal physiological metrics, or unauthorized occupancy. In addition, the command may activate audible and visual alarms within the vehicle, such as sounding the horn, flashing hazard lights, or displaying warning messages on the dashboard interface. The system may also be configured to escalate the alert by contacting emergency services or designated third parties if the condition persists or worsens, using pre-stored contact information and geolocation data. Each alert can be generated in accordance with predefined thresholds and escalation protocols, ensuring that notifications are both timely and contextually appropriate.

In some implementations, rather than using a model configured to identify the pattern of declining health, the model may be trained to generate the set of commands itself. The vector may be input into a machine learning model, such as a deep neural network or ensemble classifier, which has been trained on extensive historical datasets containing labeled examples of sensor data and corresponding system actions. The model can be configured to analyze the input vector and compare a subset of health-indicative metrics (e.g., such as heart rate variability, body temperature trends, and activity level) to a plurality of predefined patterns representing various health states. Specifically, the model may determine whether the current metrics match a pattern of declining health, such as a progressive increase in heart rate coupled with a decrease in activity and rising body temperature, as defined in the training data. Upon identifying a match to a declining health pattern, the model may generate a set of commands tailored to the detected condition, which may include actuating vehicle components (e.g., activating climate control, unlocking doors), generating alerts to user devices or emergency contacts, and initiating enhanced monitoring protocols. This process enables the system to deliver real-time, context-sensitive interventions that proactively address occupant safety and health risks.

Alternatively, or in addition to generating commands based on pattern recognition, the system may be configured to identify the commands from a plurality of available commands. The identification process may comprise evaluating the current sensor data and operational context against a command library or decision matrix, selecting the command that best addresses the detected condition (e.g., such as activating climate control, engaging door locks, or inhibiting engine start). Concurrently, or in series, the system may identify a second command from the plurality of commands, wherein the second command is configured to generate an alert at the paired user device of the primary user or to one or more additional entities located within a threshold distance of the vehicle. This may include transmitting push notifications, SMS messages, or automated calls to mobile devices, as well as activating audible or visual alarms within the vehicle. The identification of these commands may be implemented using rule-based algorithms, machine learning inference, or a combination thereof, ensuring that both vehicle actuation and alert generation are contextually appropriate and responsive to real-time sensor inputs.

In one or more embodiments, a user device—such as a smartphone, tablet, or wearable computing device—may be securely paired to the vehicle and/or the entity monitoring system to enable bidirectional communication, remote monitoring, and command execution. The pairing process may begin with the initiation of a secure handshake protocol between the user device and the vehicle's onboard telematics module or the entity monitoring system. This may be accomplished using wireless communication technologies such as Bluetooth Low Energy (BLE), Wi-Fi Direct, or Near Field Communication (NFC), depending on the hardware capabilities of both the vehicle and the user device.

During the initial pairing sequence, the user device may transmit a unique device identifier and authentication credentials, such as a cryptographically signed token or biometric verification, to the vehicle or monitoring system. The system can validate these credentials against a pre-established whitelist or via a cloud-based authentication service, ensuring that only authorized devices are permitted to pair. Upon successful authentication, a secure communication channel may be established using industry-standard encryption protocols (e.g., AES-256, TLS 1.3), and a session key can be negotiated for ongoing data exchange.

FIG. 3B illustrates an exemplary paired user device, which may display a graphical user interface (GUI) for real-time status monitoring, alert reception, and remote command input. The paired device is registered within the system's device registry, associating it with the primary user profile and enabling context-aware notifications and interventions. The system may periodically verify the proximity of the paired device using signal strength measurements, geolocation data, or periodic challenge-response exchanges, ensuring that the device remains within a defined threshold distance of the vehicle for safety-critical operations.

As described herein, the second command may cause a warning such as warning 350 to be displayed at the user device. In some implementations, the second command may also cause one or more user-interactable elements to be displayed on the device. A user interaction at the element (e.g., element 360) may cause emergency services to be called, or can be used for tracking or manually changing the vehicle operational state.

Additionally, the entity monitoring system may support multi-device pairing, allowing multiple caregivers or authorized users to receive alerts and issue commands. Device pairing status, session logs, and security events are recorded in a secure audit trail, supporting regulatory compliance and post-event analysis. This robust pairing architecture ensures that user devices are seamlessly integrated into the vehicle's safety and monitoring ecosystem, enabling real-time, context-sensitive communication and control.

According to one implementation, a command at the user device may be transmitted first to cause a notification to the user. If the primary user fails to respond or interact with the vehicle, emergency personnel may be alerted immediately. For example, when carbon monoxide is detected with a driver within the vehicle and no response on the app or vehicle dash is given, emergency personnel may be alerted immediately, preventing carbon monoxide poisonings and deaths. If a response is made, there may be a delay added before emergency personnel are alerted. In some examples, the vehicle and/or detachable monitoring system is equipped with an integrated Global Positioning System (GPS) module configured to transmit real-time location data to emergency services in the event of a detected emergency. Upon activation of an emergency protocol, such as by sensor detection of occupant distress, manual user input, or remote command from authorized personnel, the system may automatically communicate the precise geolocation coordinates of the car seat to designated emergency response centers via secure cellular or satellite communication channels. This rapid transmission of location data enables emergency personnel to pinpoint the seat position with high accuracy, significantly reducing response times and facilitating prompt intervention.

Additionally, the system may include a remote-control interface accessible to emergency personnel, which, when activated, triggers a siren and flashing lights integrated into the car seat or vehicle. This visual and audible signaling may aid emergency responders in quickly locating the car seat within crowded or complex environments, such as parking structures or public spaces. The remote control may be accessed through a secure application or dedicated hardware device, with authentication protocols in place to prevent unauthorized use.

Further, the system may be configured to assist users in locating the car seat under various scenarios, including incidents of kidnapping, carjacking, or simply forgetting the vehicle's parked location. An application executing on a user device or emergency services device may leverage the GPS module to display the real-time position of the car seat on a digital map, providing turn-by-turn navigation and proximity alerts. In cases of suspected abduction or theft, the app may escalate alerts to law enforcement and authorized caregivers, sharing location data and enabling coordinated recovery efforts.

In one or more embodiments, the vehicle safety system incorporates an integrated carbon monoxide (CO) detector configured to continuously monitor ambient air quality within the vehicle cabin, particularly when the vehicle is located in an enclosed space such as a garage. Upon detection of carbon monoxide levels exceeding a predefined safety threshold, the system may automatically initiate a shutdown sequence for the vehicle's engine to prevent further accumulation of toxic gases. Simultaneously, the system may generate an immediate alert to the parents'or caregivers'paired user devices, notifying them of the hazardous condition and advising prompt action.

Additionally, the system can be equipped with temperature sensors and physiological monitors embedded within the car seat to track both environmental and occupant health metrics. If the cabin temperature rises to a dangerous level, or if the child's oxygen saturation falls below a healthy threshold, the system may trigger an urgent alert protocol. This protocol may be designed to override silent and do not disturb settings on the paired user devices, ensuring that caregivers receive a high-priority notification regardless of device status. The alert message can provide specific instructions to remove the child from the seat and evacuate the vehicle to a safe environment. If the parents or guardians do not respond to the alert within a predetermined time window, or if sensor data indicates that the child remains in a hazardous condition, the system may escalate its response by notifying emergency personnel. The system may transmit the vehicle's real-time GPS coordinates to emergency responders, enabling rapid location and intervention.

In some implementations, the entity monitoring system is further configured to detect acute physiological emergencies, such as choking or respiratory distress, by continuously analyzing sensor data for patterns indicative of compromised airway or abnormal breathing. The detachable monitoring system may incorporate advanced respiratory sensors, including impedance pneumography, pulse oximetry, and acoustic sensors, to monitor breathing rate, oxygen saturation, and airway sounds in real time. Upon identification of sensor metrics consistent with choking (e.g., sudden cessation of airflow, abnormal respiratory sounds, or precipitous drop in oxygen saturation) or respiratory distress (e.g., rapid, shallow breathing, or hypoxemia), the system may immediately transmit a notification to the paired user device and, if configured, to additional trusted entities or emergency services.

Upon receipt of a notification indicating symptoms of choking or respiratory distress, the paired user device may automatically trigger an interactive emergency guidance interface by executing commands to display the interface at the paired user device, a device of a trusted entity or emergency services, on a display located on the detachable monitoring system and/or of the vehicle. If the system is external or remote to the display, as described herein, the system may generate one or more commands to cause the interface to be display and transmit them to the device coupled to the display.

In some implementations, the interface may be designed to provide step-by-step instructions for administering CPR and first aid, tailored to the age and size of the monitored entity. The guidance may include visual diagrams, audio prompts, and real-time feedback based on sensor data, such as confirming the effectiveness of chest compressions or rescue breaths. In some embodiments, the system may use machine learning algorithms to adapt the guidance in response to ongoing sensor readings, ensuring that caregivers receive contextually relevant and actionable instructions throughout the emergency.

Additionally, the entity monitoring system may be configured to escalate alerts based on the severity and persistence of detected symptoms. If the system determines that the monitored entity remains in distress despite caregiver intervention, or if sensor data indicate a worsening condition (e.g., persistent hypoxemia, absence of effective breathing), the system may automatically transmit the monitored entity's real-time location and health status to emergency services. This escalation protocol may include transmission of critical sensor data, such as heart rate, oxygen saturation, and recent intervention attempts, to facilitate rapid triage and targeted response by first responders. The system may also activate audible and visual alarms within the vehicle to attract attention from nearby individuals, further increasing the likelihood of timely assistance.

In some implementations, the system may notify emergency personnel, such as through a device of one or more emergency personnel or operators. In one example, the system may determine a level of emergency based on physiological symptoms. For example, a child choking may trigger a notification that is indicative of a higher level of emergency, such as one that may result in the monitored entity's death or irreparable harm. In one example, the system may store or have access to various identifiers published by or pushed to the system by emergency personnel. In particular, the system may perform one or more web crawling and extraction operations to learn the specific identifiers (e.g., codes) used by EMT services, medical services, firefighters, police, etc. Certain codes or identifiers may be associated with elevated emergency response protocols, such as expedited response times, dispatch of additional personnel or units, or prioritization over other concurrent emergency calls. For example, codes indicating life-threatening conditions may trigger a faster response, deployment of a greater number of emergency responders, or dispatch of specialized units such as advanced life support teams. In one embodiment, the system may access stored identifiers at a remote database, where the remote database is communicatively coupled to a device that may have accessed the identifiers or codes, such as through web crawling, extraction, requests to emergency personnel devices, etc.

In some implementations, the system may generate and maintain detailed logs to document the sequence of events, actions, and responses associated with an emergency situation. These logs may serve to protect both the responding emergency personnel and the parent or caregiver by providing an objective record of the circumstances surrounding the incident. For example, the system may record timestamps indicating when physiological symptoms were first detected, when notifications were transmitted to emergency services, when the parent or caregiver was alerted, and when various parties acknowledged or responded to the alerts. The system may also log actions taken by the parent or caregiver, such as attempts to intervene, interactions with the monitoring system interface, or communications initiated through the system. Similarly, the system may capture data regarding the response of emergency personnel, including dispatch times, arrival times, commands issued through the remote interface, and any alarm activations or deactivations performed by the officers. In some aspects, the logs may include sensor data, video or audio recordings where available and permitted, and metadata associated with the monitored individual's condition throughout the incident. These logs may be stored locally on the monitoring system, transmitted to a cloud-based storage system, or both, and may be encrypted or otherwise secured to preserve data integrity and confidentiality. The logged information may be made available for subsequent review by authorized parties, such as for incident reconstruction, liability assessment, quality assurance purposes, or legal proceedings, thereby providing documentation that may help establish the actions and response times of all parties involved.

In some implementations, the emergency personnel may be enabled to remotely control the vehicle, such as by communicating with a device linked to a vehicle manufacturer, e.g., such as a vehicle manufacturer that is linked to the specific vehicle the system is used with. Alternatively, or additionally, the emergency personnel may be given remote access to the user paired device, and the remote access can be used to remotely control the vehicle, such as to modify the climate, or cause the vehicle doors to unlock or open. In one example, the system may provide recommendations as part of the notification to emergency personnel where the recommendations comprise one or more sequential steps for making the state of the monitored entity safer, e.g., such as stabilizing the physiological symptoms of the monitored entity. In some examples, the system may continue to monitor during this process (e.g., during implementation of the steps by emergency personnel) and may pass along updated physiological data as real-time feedback to the emergency personnel. According to some implementations, the system may identify and notify more than one emergency personnel device, such as based on the physiological symptoms. For example, if medical services are needed, but police or firefighters are also needed to open the vehicle, the system may notify both.

The emergency personnel may be enabled to trigger alarms in the vehicle, the paired device, and/or the detachable monitoring system, as described herein. For example, haptic, auditory, and/or visual alarms may be activated to alert the driver, passengers, or bystanders of an emergency situation. The alarm triggering functionality may be implemented through various technical mechanisms. In some aspects, the emergency personnel may access a remote interface, such as a web-based dashboard, mobile application, or dedicated dispatch console, that communicates with the vehicle or detachable monitoring system via a wireless communication link. The communication link may utilize cellular networks, satellite connections, or other suitable wireless protocols to transmit alarm activation commands from the emergency personnel to the system.

Upon receiving an alarm activation command, the system may process the command through a controller or processor that interfaces with one or more alarm output devices. For auditory alarms, the system may activate speakers or buzzers within the vehicle cabin to produce alert tones, voice announcements, or siren-like sounds at configurable volume levels. Visual alarms may be implemented by activating interior lighting elements, such as LED strips or dome lights, which may flash in predetermined patterns or colors to attract attention. In some cases, the system may interface with the vehicle's existing lighting systems, such as hazard lights or headlights, to provide external visual alerts that may assist emergency responders in locating the vehicle. Haptic alarms may be delivered through vibration alerts transmitted to a paired user device, such as a smartphone or wearable device associated with a parent or caregiver. The haptic feedback may vary in intensity, duration, and pattern based on the urgency level specified by the emergency personnel.

In some implementations, the system may support simultaneous activation of multiple alarm modalities, or the emergency personnel may selectively activate specific alarm types based on the circumstances of the emergency situation. However, in some cases, triggering alarms may not be desirable or may even be contraindicated. The system may be configured to recommend whether or not to trigger an alarm based on the type of symptoms detected and the characteristics of the individual being monitored. For example, certain physiological symptoms, such as anxiety-related conditions, panic attacks, sensory processing disorders, or seizure activity, may be exacerbated by sudden auditory or visual stimuli. In particular, when monitoring children, triggering loud alarms or flashing lights may actually worsen the child's condition, increase stress levels, or induce further physiological distress. Accordingly, the system may analyze the detected symptoms and provide recommendations to emergency personnel regarding whether alarm activation is appropriate, suggest alternative notification methods such as silent alerts or gentle haptic feedback, or automatically suppress certain alarm types based on the monitored individual's profile and current physiological state.

In one or more embodiments, the system may support post-event analysis and continuous improvement of emergency response protocols. For example, some or all sensor data, notifications, user interactions, and system responses during a choking or respiratory distress event may be securely logged and anonymized for review. This data can be used to refine detection algorithms, enhance the accuracy of emergency guidance, and support regulatory compliance and quality assurance initiatives. By integrating real-time detection, interactive guidance, and automated escalation, the disclosed system may provide a comprehensive solution for managing acute health emergencies in vulnerable populations, setting a new standard for safety in sensor-based entity monitoring.

In some implementations, the system may generate and maintain detailed logs to document the sequence of events, actions, and responses associated with an emergency situation. These logs may serve to protect both the responding emergency personnel and the parent or caregiver by providing an objective record of the circumstances surrounding the incident. For example, the system may record timestamps indicating when physiological symptoms were first detected, when notifications were transmitted to emergency services, when the parent or caregiver was alerted, and when various parties acknowledged or responded to the alerts. The system may also log actions taken by the parent or caregiver, such as attempts to intervene, interactions with the monitoring system interface, or communications initiated through the system. Similarly, the system may capture data regarding the response of emergency personnel, including dispatch times, arrival times, commands issued through the remote interface, and any alarm activations or deactivations performed by the officers. In some aspects, the logs may include sensor data, video or audio recordings where available and permitted, and metadata associated with the monitored individual's condition throughout the incident. These logs may be stored locally on the monitoring system, transmitted to a cloud-based storage system, or both, and may be encrypted or otherwise secured to preserve data integrity and confidentiality. The logged information may be made available for subsequent review by authorized parties, such as for incident reconstruction, liability assessment, quality assurance purposes, or legal proceedings, thereby providing documentation that may help establish the actions and response times of all parties involved.

In some implementations, the system may incorporate safeguards to protect against unauthorized access, nefarious intent, or accidental misuse of the monitoring application. For example, even if a person physically present at the vehicle interacts with the application interface, such as by tapping buttons or attempting to dismiss alerts, the system may require additional authentication before allowing certain actions to be completed. In some aspects, the system may require biometric authentication, such as fingerprint scanning, facial recognition, or voice recognition, to verify that the individual attempting to silence alarms, dismiss notifications, or mark an emergency as resolved is an authorized caregiver or parent. This may help prevent scenarios where an unauthorized individual attempts to suppress alerts while failing to actually assist the monitored occupant.

In some cases, the system may implement multi-factor authentication that combines biometric verification with a secondary confirmation, such as a PIN code, password, or response to a security question. The system may also require that certain high-stakes actions, such as canceling an emergency dispatch or disabling monitoring functionality, be confirmed through a separate trusted device associated with the registered caregiver. In some implementations, the system may analyze behavioral patterns to detect potentially suspicious activity, such as repeated failed authentication attempts, unusual interaction patterns, or attempts to disable the system during an active emergency alert. The system may also implement time-delayed confirmations for certain actions, requiring the user to maintain authentication for a predetermined period before the action takes effect, which may provide additional time for emergency personnel to assess the situation. In some aspects, the system may transmit verification requests to secondary contacts or emergency personnel when an individual attempts to dismiss alerts without proper authentication, or when the system detects anomalies in the dismissal process. The system may further maintain an audit trail of all authentication attempts and alert dismissals, which may be reviewed to identify patterns of misuse or to support subsequent investigations.

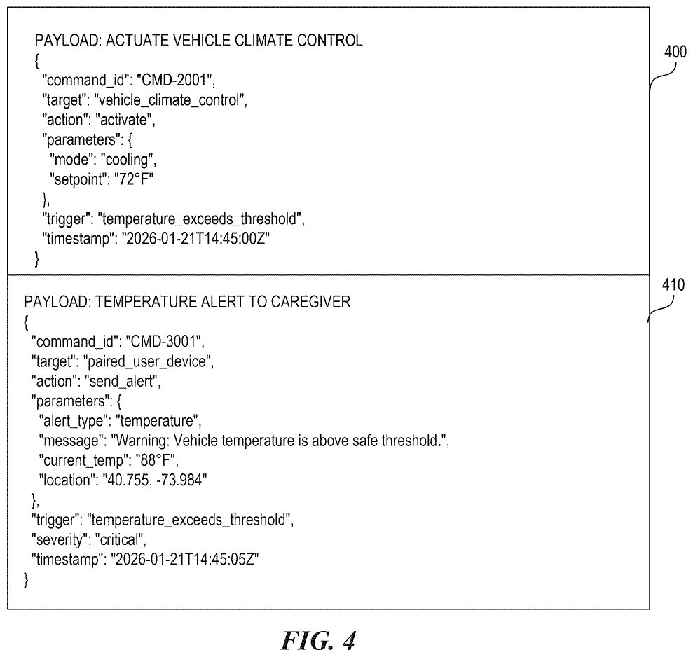

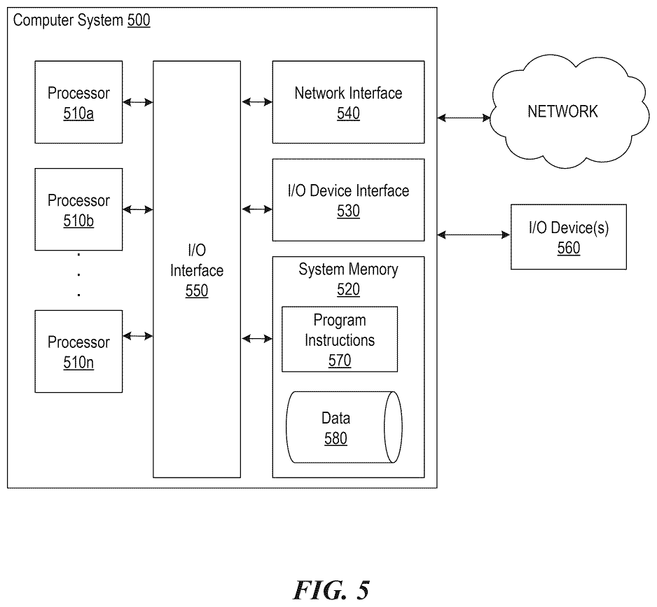

FIG. 5 shows an example computing system that may be used in accordance with some embodiments of this disclosure. In some instances, computing system 500 is referred to as a computer system 500. A person skilled in the art would understand that those terms may be used interchangeably. The components of FIG. 5 may be used to perform some or all operations discussed in relation to FIGS. 1-4. Furthermore, various portions of the systems and methods described herein may include or be executed on one or more computer systems similar to computing system 500. Further, processes and modules described herein may be executed by one or more processing systems similar to that of computing system 500.

Computing system 500 may include one or more processors (e.g., processors 510a-510n) coupled to system memory 520, an input/output (I/O) device interface 530, and a network interface 540 via an I/O interface 550. A processor may include a single processor or a plurality of processors (e.g., distributed processors). A processor may be any suitable processor capable of executing or otherwise performing instructions. A processor may include a CPU that carries out program instructions to perform the arithmetical, logical, and I/O operations of computing system 500. A processor may execute code (e.g., processor firmware, a protocol stack, a database management system, an operating system, or a combination thereof) that creates an execution environment for program instructions.

A processor may include a programmable processor. A processor may include general or special purpose microprocessors. A processor may receive instructions and data from a memory (e.g., system memory 520). Computing system 500 may be a uni-processor system including one processor (e.g., processor 510a), or a multiprocessor system including any number of suitable processors (e.g., 510a-510n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field-programmable gate array) or an ASIC (application-specific integrated circuit). Computing system 500 may include a plurality of computing devices (e.g., distributed computer systems) to implement various processing functions.

I/O device interface 530 may provide an interface for connection of one or more I/O devices 560 to computer system 500. I/O devices may include devices that receive input (e.g., from a user) or output information (e.g., to a user). I/O devices 560 may include, for example, a graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 560 may be connected to computer system 500 through a wired or wireless connection. I/O devices 560 may be connected to computer system 500 from a remote location. I/O devices 560 located on remote computer systems, for example, may be connected to computer system 500 via a network and network interface 540.

The I/O device interface 530 and I/O devices 560 may be used to enable manipulation of the three-dimensional model as well. For example, the user may be able to use I/O devices such as a keyboard and touchpad to indicate specific selections for nodes, adjust values for nodes, select from the history of machine learning models, select specific inputs or outputs, and/or the like. Alternatively or additionally, the user may use their voice to indicate specific nodes, specific models, and/or the like via the voice recognition device and/or microphones.

Network interface 540 may include a network adapter that provides for connection of computer system 500 to a network. Network interface 540 may facilitate data exchange between computer system 500 and other devices connected to the network. Network interface 540 may support wired or wireless communication. The network may include an electronic communication network, such as the internet, a LAN, a WAN, a cellular communications network, or the like.

System memory 520 may be configured to store program instructions 570 or data 580. Program instructions 570 may be executable by a processor (e.g., one or more of processors 510a-510n) to implement one or more embodiments of the present techniques. Program instructions 570 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages.

A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program may be deployed to be executed on one or more computer processors located locally at one site or distributed across multiple remote sites and interconnected by a communication network.

System memory 520 may include a tangible program carrier having program instructions stored thereon. A tangible program carrier may include a non-transitory, computer-readable storage medium. A non-transitory, computer-readable storage medium may include a machine-readable storage device, a machine-readable storage substrate, a memory device, or any combination thereof. A non-transitory, computer-readable storage medium may include non-volatile memory (e.g., flash memory, ROM, PROM, EPROM, EEPROM), volatile memory (e.g., random access memory (RAM), static random access memory (SRAM), synchronous dynamic RAM (SDRAM)), bulk storage memory (e.g., CD-ROM and/or DVD-ROM, hard drives), or the like. System memory 520 may include a non-transitory, computer-readable storage medium that may have program instructions stored thereon that are executable by a computer processor (e.g., one or more of processors 510a-510n) to cause the subject matter and the functional operations described herein. A memory (e.g., system memory 520) may include a single memory device and/or a plurality of memory devices (e.g., distributed memory devices).

I/O interface 550 may be configured to coordinate I/O traffic between processors 510a-510n, system memory 520, network interface 540, I/O devices 560, and/or other peripheral devices. I/O interface 550 may perform protocol, timing, or other data transformations to convert data signals from one component (e.g., system memory 520) into a format suitable for use by another component (e.g., processors 510a-510n). I/O interface 550 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 500 or multiple computer systems 500 configured to host different portions or instances of embodiments. Multiple computer systems 500 may provide for parallel or sequential processing/execution of one or more portions of the techniques described herein.

Those skilled in the art will appreciate that computer system 500 is merely illustrative and is not intended to limit the scope of the techniques described herein. Computer system 500 may include any combination of devices or software that may perform or otherwise provide for the performance of the techniques described herein. For example, computer system 500 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, a Global Positioning System (GPS), or the like. Computer system 500 may also be connected to other devices that are not illustrated or may operate as a stand-alone system. In addition, the functionality provided by the illustrated components may, in some embodiments, be combined in fewer components, or be distributed in additional components. Similarly, in some embodiments, the functionality of some of the illustrated components may not be provided, or other additional functionality may be available.

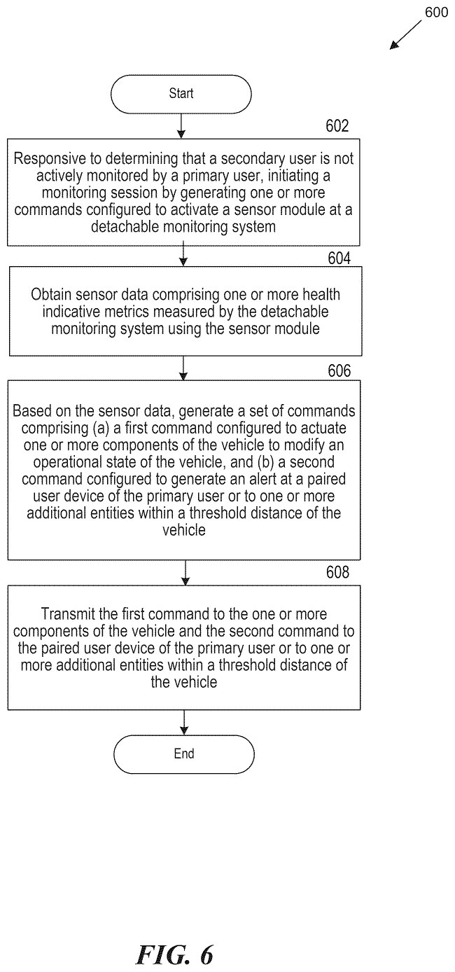

FIG. 6 is a flowchart 600 of operations for remote monitoring of users at a vehicle, in accordance with one or more embodiments of this disclosure. The operations of FIG. 6 may use components described in relation to FIG. 5. In some embodiments, entity monitoring system 160 may include one or more components of computer system 500.

At operation 602, one or more of processors 510a-510n may be used to initiate a monitoring session by generating one or more commands configured to activate a sensor module at a detachable monitoring system responsive to determining that a secondary user is not actively monitored by a primary user.

At operation 604, one or more of processors 510a-510n may obtain sensor data comprising one or more health indicative metrics measured by the detachable monitoring system using the sensor module. One or more of processors 510a-510n may receive such sensor data over network 140 using network interface 540. Alternatively, or additionally, depending on where the entity monitoring system is hosted or executed on, the sensor data may be obtained locally.

At operation 606, based on the sensor data, the one or more of processors 510a-510n may generate a set of commands comprising (a) a first command configured to actuate one or more components of the vehicle to modify an operational state of the vehicle, and (b) a second command configured to generate an alert at a paired user device of the primary user or to one or more additional entities within a threshold distance of the vehicle.

At operation 608, one or more of processors 510a-510n may transmit the first command to the one or more components of the vehicle and the second command to the paired user device of the primary user or to one or more additional entities within a threshold distance of the vehicle.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The above-described embodiments of the present disclosure are presented for purposes of illustration, not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

What is claimed is:

1. A system for remote monitoring of users at a vehicle, the system comprising:
one or more processors; and
one or more memories configured to store instructions that, when executed by the one or more processors, perform operations comprising:
obtaining a location of a detachable monitoring system in relation with the vehicle, wherein the detachable monitoring system is configured to measure, via a sensor module of the detachable monitoring system, one or more health indicative metrics of a secondary user while a secondary user is engaged with the detachable monitoring system;
responsive to determining that (i) the detachable monitoring system is located within the vehicle and (ii) that the secondary user is not actively monitored by a primary user, initiating a monitoring session by generating one or more commands configured to activate the sensor module at the detachable monitoring system;
obtaining sensor data comprising one or more health indicative metrics measured by the detachable monitoring system using the sensor module;
based on the sensor data, generating a set of commands comprising (a) a first command configured to actuate one or more components of the vehicle to modify an operational state of the vehicle, and (b) a second command configured to generate an alert at a paired user device of the primary user or to one or more additional entities within a threshold distance of the vehicle; and
transmitting the first command to the one or more components of the vehicle and the second command to the paired user device of the primary user or to one or more additional entities.

2. The system of claim 1, wherein determining that the secondary user is not actively monitored by the primary user comprises (a) detecting that a paired user device of a primary user is outside of a predetermined distance from the vehicle or (b) that the vehicle is in operation.

3. The system of claim 1, wherein the instructions further cause the one or more processors to perform operations comprising:
detecting that the paired user device of a primary user is outside of a predetermined distance from the vehicle; and
responsive to detecting that the paired user device is outside of the predetermined distance, modifying the operational state of the vehicle to maintain temperature of the vehicle within an upper threshold temperature and a lower threshold temperature.

4. The system of claim 1, wherein generating the set of commands comprises:
generating, using the sensor data, a vector for input to a machine learning model trained to generate the set of commands; and
inputting the vector into the machine learning model to obtain the set of commands.

5. The system of claim 1, wherein generating the set of commands based on the sensor data comprises:
determining that a subset of health indicative metrics matches a pattern of declining health from a plurality of patterns;
identifying the first command configured to actuate one or more components of the vehicle to modify the operational state of the vehicle from a plurality of commands; and
identifying the second command configured to generate the alert at the paired user device of the primary user or to one or more additional entities within the threshold distance of the vehicle from the plurality of commands.

6. The system of claim 1, wherein the instructions further cause the one or more processors to perform operations comprising:
determining that the primary user is attempting to operate the vehicle;
determining (i) whether the detachable monitoring system is within a vehicle and (ii) whether the secondary user is engaged with the detachable monitoring system; and
transmitting a command configured to generate an alert at the paired user device responsive to determining that the detachable monitoring system is not within a vehicle or that the secondary user is not engaged with the detachable monitoring system.

7. A method for remote monitoring of users at a vehicle, the method comprising:
responsive to determining that a secondary user is not actively monitored by a primary user, initiating a monitoring session by generating one or more commands configured to activate a sensor module at a detachable monitoring system;
obtaining sensor data comprising one or more health indicative metrics measured by the detachable monitoring system using the sensor module;
based on the sensor data, generating a set of commands comprising (a) a first command configured to actuate one or more components of the vehicle to modify an operational state of the vehicle, and (b) a second command configured to generate an alert at a paired user device of the primary user or to one or more additional entities within a threshold distance of the vehicle; and
transmitting the first command to the one or more components of the vehicle and the second command to the paired user device of the primary user or to one or more additional entities within a threshold distance of the vehicle.

8. The method of claim 7, further comprising: obtaining a location of a detachable monitoring system in relation with the vehicle.

9. The method of claim 7, wherein determining that the secondary user is not actively monitored by the primary user comprises (a) detecting that a paired user device of a primary user is outside of a predetermined distance from the vehicle or (b) that the vehicle is in operation.

10. The method of claim 7, further comprising:
detecting that the paired user device of a primary user is outside of a predetermined distance from the vehicle; and
responsive to detecting that the paired user device is outside of the predetermined distance, modifying the operational state of the vehicle to maintain temperature of the vehicle within an upper threshold temperature and a lower threshold temperature.

11. The method of claim 7, wherein generating the set of commands comprises:
generating, using the sensor data, a vector for input to a machine learning model trained to generate the set of commands; and
inputting the vector into the machine learning model to obtain the set of commands.

12. The method of claim 7, wherein generating the set of commands based on the sensor data comprises:
determining that a subset of health indicative metrics matches a pattern of declining health from a plurality of patterns;
identifying the first command configured to actuate one or more components of the vehicle to modify the operational state of the vehicle from a plurality of commands; and
identifying the second command configured to generate the alert at the paired user device of the primary user or to one or more additional entities within the threshold distance of the vehicle from the plurality of commands.

13. The method of claim 7, further comprising:
determining that the primary user is attempting to operate the vehicle;
determining (i) whether the detachable monitoring system is within a vehicle and (ii) whether the secondary user is engaged with the detachable monitoring system; and
transmitting a command configured to generate an alert at the paired user device responsive to determining that the detachable monitoring system is not within a vehicle or that the secondary user is not engaged with the detachable monitoring system.

14. The method of claim 7, wherein the one or more additional entities comprises an emergency services entity.

15. One or more non-transitory, computer-readable storage media storing instructions that when executed by one or more processors cause the one or more processors to perform operations comprising:
responsive to determining that a detachable monitoring system is located within a vehicle, initiating a monitoring session by generating one or more commands configured to activate a sensor module at the detachable monitoring system;
obtaining sensor data comprising one or more health indicative metrics of a secondary user measured by the detachable monitoring system using the sensor module;
based on the sensor data, generating a set of commands comprising (a) a first command configured to actuate one or more components of the vehicle to modify an operational state of the vehicle, and (b) a second command configured to generate an alert at a paired user device of a primary user or to one or more additional entities within a threshold distance of the vehicle; and
transmitting the first command to the one or more components of the vehicle and the second command to the paired user device of the primary user or to one or more additional entities.

16. The one or more non-transitory, computer-readable storage media of claim 15, wherein the instructions further cause the one or more processors to perform operations comprising:
obtaining a location of a detachable monitoring system in relation with the vehicle, wherein the detachable monitoring system is configured to measure, via a sensor module of the detachable monitoring system, one or more health indicative metrics of a secondary user while a secondary user is engaged with the detachable monitoring system.

17. The one or more non-transitory, computer-readable storage media of claim 15, wherein the instructions further cause the one or more processors to perform operations comprising:
determining that the secondary user is not actively monitored by a primary user based on detecting (a) that a paired user device of a primary user is outside of a predetermined distance from the vehicle or (b) that the vehicle is in operation; and
responsive to determining that the secondary user is not actively monitored by a primary user, activating a sensor module at the detachable monitoring system.

18. The one or more non-transitory, computer-readable storage media of claim 15, wherein the instructions further cause the one or more processors to perform operations comprising:
detecting that the paired user device of a primary user is outside of a predetermined distance from the vehicle; and
responsive to detecting that the paired user device is outside of the predetermined distance, modifying the operational state of the vehicle to maintain temperature of the vehicle within an upper threshold temperature and a lower threshold temperature.

19. The one or more non-transitory, computer-readable storage media of claim 15, wherein generating the set of commands based on the sensor data comprises:
determining that a subset of health indicative metrics matches a pattern of declining health from a plurality of patterns;
identifying the first command configured to actuate one or more components of the vehicle to modify the operational state of the vehicle from a plurality of commands; and
identifying the second command configured to generate the alert at the paired user device of the primary user or to one or more additional entities within the threshold distance of the vehicle from the plurality of commands.

20. The one or more non-transitory, computer-readable storage media of claim 15, wherein the instructions further cause the one or more processors to perform operations comprising:
determining that the primary user is attempting to operate the vehicle;
determining (i) whether the detachable monitoring system is within a vehicle and (ii) whether the secondary user is engaged with the detachable monitoring system; and
transmitting a command configured to generate an alert at the paired user device responsive to determining that the detachable monitoring system is not within a vehicle or that the secondary user is not engaged with the detachable monitoring system.
